(19) United States
(12) Patent Application Publication
(10) Pub. No.: US 2011/0092852 A1
(43) Pub. Date: Apr. 21, 2011

(54) DEVICE FOR ABSORBING PROTEINS FROM BODY FLUIDS

(75) Inventors: Kurt Maier, Berlin (DE); Sarvan Kumar Munjal, Mannheim (DE)

(73) Assignee: Dentognostics GmbH

(21) Appl. No.: 12/934,341

(22) PCT Filed: Mar. 30, 2009
(86) PCT No.: PCT/EP09/53751

§ 371 Date: Dec. 22, 2010

(30) Foreign Application Priority Data
Mar. 28, 2008 (EP) 08153560.1

(51) Int. Cl. A61B 5/00

(52) U.S. Cl. 600/573

(57) ABSTRACT

A planar device for absorbing substances, especially proteins, such as enzymes, from body fluids, especially from gingival crevicular fluid or lacrimal fluid, comprising a receptor element (10), a support element (20), characterized in that—said receptor element (10) is hydrophilic and has a pore size of from 0.22 μm to 5 μm, especially from 0.5 μm to 3 μm, and consists of a plastic material or mixture of plastic materials, especially of an inert plastic material or a mixture of inert plastic materials;—said support element (20) is hydrophobic and covers one surface of the receptor element (10) at least partially, and—wherein the device further comprises a discriminator element (40) which is situated at the opposing surface of the receptor element (10).

The invention relates to a device for absorbing proteins from body fluids, and to the use of that device.

Gingival crevicular fluid (GCF), saliva, lacrimal fluid or exudations of wounds are media that can be recovered in a non-invasive way and thus without surgical intervention, for example, at the dentist's, at the bedside or during police controls. In diagnostics, they can play an important role because they may contain, inter alia, human proteins and proteins from microorganisms, medicinal drugs and their metabolic products, intoxicating substances (illegal drugs) and their metabolic products, or free radicals.

Generally, the concentration of substances in non-invasively obtained fluids can be an analogous image of the levels of such substances present in the serum of mammals. Further, locally occurring substances, such as inflammation markers, can indicate local pathological alterations at the sampling site, such as inflammations of the peridontium. However, these are known (Uitto, Periodontology 2000, Vol. 31, 2003) to be present in a lower, in part substantially lower, concentration, so that highly sensitive methods are required for analysis (e.g., immunoassays, such as ELISA methods).

For example, in diagnostic or analytical determinations (assays), the concentration of such substances allows conclusions to be made relating to:
current and future diseases of the peridontium (e.g., periodontitis, peri-implantitis, root caries);
current and future general diseases (e.g., viral diseases);
current and probable systemic diseases (e.g., diabetes, allergies);
current acute diseases (infections);
the immune status (vaccinations);
risks of future diseases (free radicals, immune status).

Thus, for example, gingival crevicular fluid is examined in dental analytics, especially in the monitoring of gingivitis and periodontitis diseases. Gingivitis and periodontitis are caused by permanent challenge from the dental biofilm. However, whether a periodontitis actually occurs is determined by the defense reaction of the host organism. Host endogenous collagenases are responsible for the beginning and progression of a periodontitis and of alveolar bone destruction.

The most important collagenase for periodontopathogenic destruction processes is matrix metalloproteinase-8 (MMP-8), or collagenase 2.

Matrix metalloproteinases occur in three different forms:

1. inactive precursor or pro form (storage form in polymorphonuclear granulocytes)
2. activated or active form
3. inhibited form or complex form.

The active form released in the tissue (aMMP-8) is ultimately responsible for the tissue destruction. A high aMMP-8 level indicates an active inflammation and thus an acute state needing treatment.

aMMP-8 is an objective diagnostic marker for recognizing the time when periodontal tissue is destroyed.

According to the prior art, proteins are recovered from body fluids by simple water-absorbing membranes as receptor elements. As a rule, blotting paper strips are used. When samples are taken by means of blotting paper strips, a number of problems can occur.

Thus, when GCF is collected, the sample can be contaminated or diluted by contact with saliva or pellicles. Further, undesirable components, such as epithelial cells, mucosal cells, bacteria, PMN cells (polymorphonuclear neutrophil cells), cell components or solids, can be taken up or adhere and interfere with the later analytical determinations.

The shape and size of the membranes is often non-standardized and can thus lead to variations in the amount of sample taken up. During application, handling problems (e.g., excessively deep dipping into the liquid, insufficient and incomplete filling, mispositioned application) may lead to application errors. Further, the elution of the samples and the sought analytes contained is often incomplete, time-consuming and not standardizable.

US 2004/057876 discloses a device that absorbs preferably saliva whereas GCF without impurities cannot be absorbed. Further, this device is not planar and does not comprise a discriminator element.

EP-A-0 420 021 discloses hydrophilic laminated porous membranes which cannot be applied to tooth pockets and consequently does not absorb GCF. Since these membranes do not comprise a discriminator element, the direction of use is not defined. In addition, the membranes do not comprise a rounded end which may cause an injury in the tooth pocket.

WO 93/04193 discloses hydrophilic PVDF membranes and a plastic support but fails to disclose a device comprising at least a discriminator element or a rounded end which can absorb GCF without impurities.

U.S. Pat. No. 5,656,448 discloses an immunoassay dip stick comprising a membrane and a plastic sheet, wherein the stick does not comprise a rounded end or a discriminator element. Furthermore, this dip stick is not capable of absorbing GCF without impurities.

The transport of samples in the membranes of the prior art blotting paper strips is possible only to a limited extent, so that the samples must be further processed and frozen at the place of sampling, for example, in worldwide studies for examining GCF (Uitto, Periodontology 2000, Vol. 31, 2003).

This had also the consequence that the analysis of such samples was reserved to a few universities, and a ubiquitous utilization of GCF, for example, as a diagnostic medium has not been done or has not been possible.

Thus, the object of the invention is to provide a device for the improved absorption of substances, especially proteins, which enables the storage of the substances and further overcomes the mentioned drawbacks of the prior art receptor elements.

“Absorption” as used herein means the uptake of substances by a receptor element. This is not a deposition at the surface of the receptor element (adsorption), but an uptake into the bulk of the receptor element.

Further, the “modulus of elasticity” (Young's modulus) is a material characteristic indicating the stiffness of a material. The SI unit of the modulus of elasticity is Pascal (N/m²). The modulus of elasticity values stated in the following is for room temperature (20° C.).

The term “discriminator element” as used herein characterizes an element which is intended to define the direction of use or introduction of the device. Further, the device according to the invention is planar, in particular when it comprises two major opposing surfaces and/or it has a two-dimensional characteristic as e.g. the device according to FIGS. 1 and 2.

Further, “hydrophilicity” refers to the capability of a material to take up (absorb) water or aqueous solutions. “Hydrophobicity” is the antonym thereof and describes the tendency of a material not to absorb water or aqueous solutions, if possible.

Further, chemically “inert” relates to substances which do not or virtually not undergo a chemical reaction with other materials.

A “plastic material” refers to a material which consists of synthetically produced organic polymers. “Mixtures of plastic materials” consist of at least two plastic materials.

“Storage” means that enzymes, in particular aMMP-8, are stored for a period of time which is more than 3 or at least more than 1 day, in particular 1-31 days. During the storage period the enzymes are “stable” which means that the activity of the enzymes does not significantly decrease over the period of storage.

The invention relates e.g. to a planar device 5 for absorbing substances, especially proteins, such as enzymes, from body fluids, especially from gingival crevicular fluid (GCF) or lacrimal fluid, comprising a receptor element 10 and a support element 20, characterized in that
said receptor element 10 is hydrophilic and has a pore size of from 0.22 μm to 5 μm, especially from 0.5 μm to 3 μm, and consists of a plastic material or mixture of plastic materials, especially of an inert plastic material or a mixture of inert plastic materials;
said support element 20 is hydrophobic and covers one surface of the receptor element 10 at least partially or completely; and
wherein the device further comprises a discriminator element 40 which is situated at the opposing surface of the receptor element 10.

The discriminator element 40 is intended to indicate the direction of use of the device, since it is one advantage of the device that it can be placed with e.g. a forceps in tooth pockets. The device should be placed in the pockets such that the discriminator elements 40 face the front side, whereby wrong application can be avoided. Consequently, contamination of the sample by contact with saliva or pellicle is avoided when GCF is absorbed.

Further, the pore size precludes the absorption of whole cells and large cell components as well as microorganisms.

In one embodiment of the device 5 according to the invention, the receptor element 10 has a pore size of from 0.6 μm to 2.5 μm, especially from 0.75 μm to 1.75 μm, or from 0.9 μm to 1.35 μm.

In a further embodiment of the device 5 the discriminator element 40 is located at the opposite end of a rounded end 30 of the device. Thus, the discriminator element is clearly visible after placing the device in the tooth pocket. To indicate the direction of use, the discriminator element may be characterized by a specific colour, fluorescence or marked-up surface etc. However, all possible ways of indications can be used as discriminator element which do not emit toxic or harmful substances or other kinds of impurities.

In another embodiment, the receptor element 10 is a membrane. In a further embodiment, such membrane 10 consists of a plastic material or mixture of plastic materials, especially of an inert plastic material or mixture of inert plastic materials.

In still another embodiment of the device 5 according to the invention, the plastic material is a fluorohydrocarbon polymer, especially polyvinylidene fluoride (PVDF).

In a further embodiment, the device 5 comprising receptor element 10 and support element 20 and therefore comprises said plastic material or mixture of plastic materials which preferably has a modulus of elasticity (Young's modulus) of from 1 to 6 GPa, especially from 2 to 5, from 1 to 4, from 1 to 2, from 1 to 3 or from 2 to 3 GPa.

In a further embodiment of the invention, the support element 20 also comprises a plastic material or mixture of plastic materials whose modulus of elasticity (Young's modulus) is from 1 to 4 GPa, especially from 2 to 3, from 1 to 2 or from 1 to 3 GPa. This has the advantage that the device 5 is not deformed upon taking up the body fluid and thus can be withdrawn from the body fluid without difficulty, for example, in the gingival sulcus or in the eye of a patient. In addition, this has the advantage that only the body fluid from the gingival sulcus is taken up, and other fluids of the oral cavity, such as saliva or pellicles, are not absorbed.

In an additional embodiment as shown in FIG. 5, the device according to the invention has a rounded end 30 at the receptor element 10, and preferably also at the support element 20. This rounded end prevents the patient from being hurt by the device 5.

In one embodiment of the device 5 according to the invention, the receptor element 10 has a pore size of 1.2 μm. It can be sterilized by autoclaving at up to 135° C./3082 hPa, for 45 minutes.

In another embodiment of the device 5, it has a marking element 60 which indicates the desired immersion depth of the device in the body fluid. This simplifies the handling of device 5.

In another embodiment of the device according to the invention, the color element 40 which designates the surface of the receptor element 10 facing away from the support element 20 is provided opposite to the rounded end 30. This facilitates the correct handling of device 5, because both the receptor side and the front and back sides of the device are thus determined (see FIG. 1-5).

Another aspect of the invention is the use of device 5 for the absorption and/or storage of substances, especially proteins, such as enzymes, especially collagenases, such as matrix metalloproteinase-8 (MMP-8), MMP-13, TNFα, Interleukin 1β (IL-1β), Osteoprotegerin, from body fluids, especially from gingival crevicular fluid or lacrimal fluid. The enzymes, such as matrix metalloproteinases, are absorbed in their inactive precursor form (zymogen etc.), activated form or inhibited form. Further, antibodies, bacterial proteins or free radicals are also absorbed. These absorbed substances can be assayed analytically or used in another way scientifically or technically, optionally after storage. An analysis may also be performed directly on or in the device 5.

In one embodiment of such use of device 5, the enzymes are stable over a storage time of from 1 to 31 days, especially from 7 to 31 or from 7 to 21 days, at a temperature of from 4° C. to 42° C., especially from 4° C. to 37° C., or from 8° C. to 20° C. This enables a simple storage even at room temperature and further a simple shipping of the device according to the invention including absorbed substances.

After such storage, the activity of the enzymes is even approximately or actually maintained. This proves that the activity of the enzymes does not decrease during storage, but is preserved by the device 5.

A further embodiment of the invention is the use of device 5 for stabilizing proteins, especially enzymes, characterized in that the immune reactivity of the enzymes, especially the epitopes of the enzymes is approximately maintained after the storage.

### DESCRIPTION OF THE FIGURES

FIG. 1 shows an example of the device 5 according to the invention in cross-sectional view. The receptor element 10 is attached to the support element 20. At one end, the device 5 has a rounded end 30 which draws in the body fluid. At the opposite end, the discriminator element 40 is provided which designates the surface of the receptor element 10 facing away from the support element 20. Further shown is the indicator element 50 which indicates the sufficient filling of the receptor element 10. The marking element 60 indicates the desired immersion depth of the device 5 in the body fluid. In addition, the device 5 is attached to the bottom element 70 through the attachment material 80.

FIG. 2 shows an example of the device 5 according to the invention in a top plan view. The receptor element 10 according to the invention comprises the rounded end 30 and the discriminator element 40. Further shown is the marking element 60.

FIG. 3 is a top plan view showing how several devices 5 are provided on the bottom element 70.

FIG. 4 is another cross-sectional view with the dimensions of one embodiment of the device 5 according to the invention which is attached to the bottom element 70 through the attachment material 80.

FIG. 5 discloses a top plan view of the design of the device 5 according to the invention with a receptor element 10, rounded end 30 and color element 40.

FIGS. 6a and 6b disclose the concentration of aMMP-8 in the absorbed device incubated at room temperature (RT) and 37° C., and also the positive controls incubated at room temperature (RT-K) and 37° C. (37° C.-K).

FIG. 7 depicts a constant amount of aMMP-8 in the absorbed strips over a period of 5 days with fluctuation in temperature from 4° C. to 42° C.

FIGS. 8a and 8b: The concentrations of aMMP-8 recovered from the absorbed strips at day 0 and 14 incubated by RT and 4° C. are shown.

FIGS. 9a and 9b: The concentrations of aMMP-8 quantified using densitometry in the absorbed strips at day 0 and day 14 incubated by RT and 4° C. are shown.

FIGS. 10a and 10b disclose the concentrations of the activated form of MMP-9 quantified using densitometry in the absorbed strips at day 0 and day 14 incubated by RT and 4° C.

FIGS. 11a and 11b disclose the concentrations of myeloperoxidase (MPO) quantified using ELISA in the absorbed strips at day 0 and day 14 incubated by RT and 4° C.

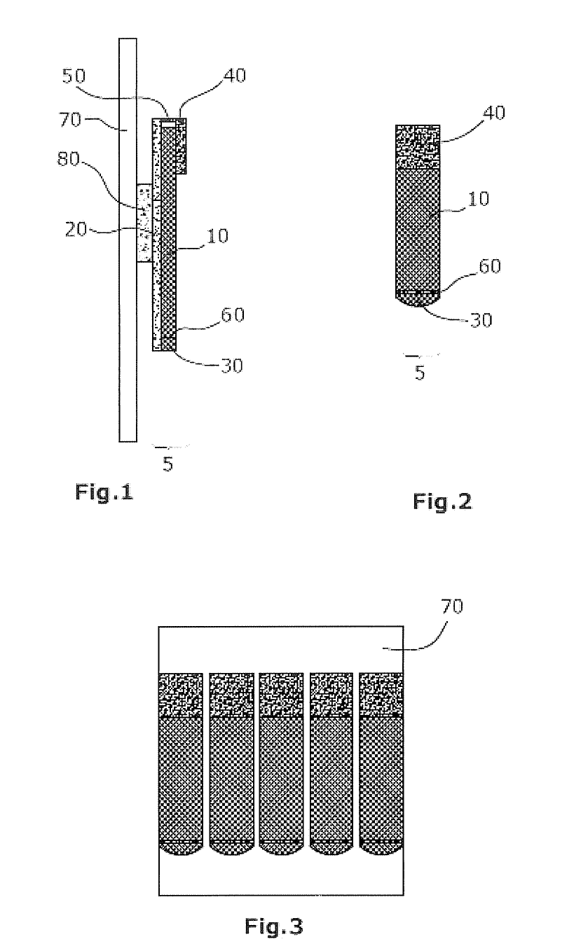

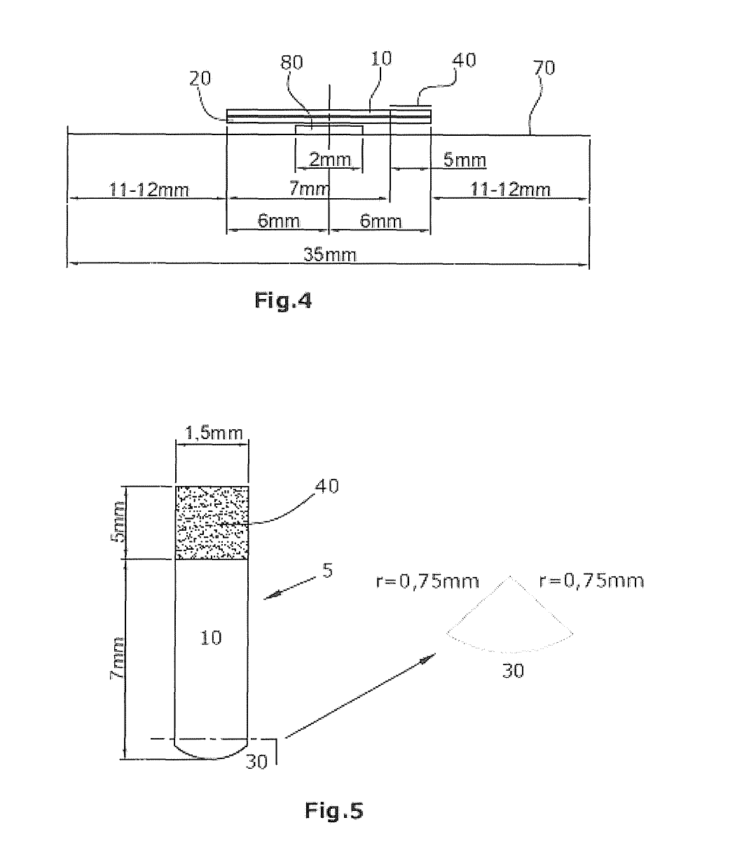

### EXAMPLES

### Example 1

### Specification of Hydrophilic Receptor Element

The membrane is cast as an integral, homogeneous film. The membrane may be sterilized by autoclaving up to 135° C., 30 psig for 45 minutes. Integrity of the membrane may be determined by the bubble point test.

| PROPERTY | PRODUCT DESIGN |
|---|---|
| Materials | The membrane is made of modified |
| | polyvinylidene fluoride. |
| Measurements | Rollstock width is ≧280 mm |
| Thickness | 90 μm ≦ × ≦ 140 μm |
| Bubble Point (H₂0) | 8 hPa ≦ × ≦ 13 hPa |
| Flow Time (H₂0) | x ≦ 20 sec. at 25° C., 1.9 bar (500 ml |
| | through a 47 mm disc) |
| Pyrogenicity | <0.5 Eu/ml E. Coli reference endotoxin |
| | (BVPP00000 only) |
| Shrinkage | ≦2.2% with no value >2.7% at 126° C. for |
| | 45 minutes. |
| Wettability | Filter wets completely in <30 seconds in |
| | 10 wt. % NaCl (Aq.) |
| Strength | X ≧ 771 g |
| Elongation | x ≧ 15% |
| Extractables | Oxidizable Substance: The effluent must |
| | pass the current USP test for oxidizable |
| | substance after 100 ml WFI flush (47 mm). |
| | Gravimetric extractables ≦0.50 weight % |
| | by Methanol Soxhlet. |
| Toxicity | The membrane is manufactured to pass |
| | the current edition USP mouse safety test. |

### Example 2

### Specification of Device

Bottom Element 70: Melinex 539 Polyester film (175 μm) manufactured by ICI.
Attachment material 80: Medical grade adhesive

### Support Element 20:

Product No: ARCARE 7815
Glue: AS110, acrylic Medical Grade Adhesive
Substrate: 51 μm polyethylene film (PET)
Coating: Silicon PET coating
Discriminator Element 40: coloured film

### Example 3

### Method of Sample Collection and Elution

The GCF samples were collected according to standard method by placing a GCF collection strip with a forceps in tooth pockets for 30 s. The GCF strips should be placed in the pockets such that the discriminator elements 40 face the front side and only 2-3 mm of the strips remain in the pocket. After 30 s, the strips containing GCF are placed in 1.5 ml eppendorf tube.

Elution: The elution buffer (containing 15 mM Na₂HPO₄*12 H₂O, 7 mM NaH₂PO₄*H₂O, 550 nM NaCl, 0.05% 5-bromo-5-nitro-1,3-dioxane (BND), 0.2% bovine serum albumin (BSA) and 0.3% Tween 20 (Polysorbat 20) or Tetronic 1307 (BASF SE, block copolymer of ethylene oxide/propylene oxide)) was poured using a pipette in the eppendorf tubes containing the strips and incubated for 5 min at room temperature. The tubes were manually inverted for at least 5 times before gently removing the strips with a forceps. The samples (after elution) should be either analyzed immediately or stored at −20° C.

The samples were analyzed quantitatively in a MMP-8 sensitive ELISA (Enzyme-linked immunosorbent assay). The eluate was further analyzed according to the method of Prescher et al. and Munjal et al. (Prescher et al., Ann N Y Acad Sci, 2007, 1098, 493-95; Munjal et al., Ann N Y Acad Sci, 2007, 1098, 490-92).

### Example 4

### ELISA for Quantitative Detection of aMMP-8

Procedure: ELISA was carried out according to Manufacturer's instructions (dentoELISA, dentognostics GmbH, Jena, Germany). The ELISA was based on sandwich immunoassay system using two specific monoclonal (mab) antibodies (K8708 and K8706) (Medix Biochemica, Finland) against aMMP-8 (Hanemaaijer et al 1997). Briefly, 96-well, flat-bottom plates (Nunc) were coated with mab K8708 at a concentration of 1 μg/ml using an automated ELISA coating platform (Seramun, Germany). A clinical sample was prepared by diluting 1:50 in dilution-buffer. All standards and controls were prepared as per the Instruction's manual of the ELISA. 100 μl of standards, controls and diluted clinical samples was dispensed into appropriate wells in duplicates. The plate was covered with foil and incubated at 37° C. for 1 hour. The plate was washed 5 times with washing buffer using automatic washer. The detection antibody (K8706) conjugated with poly-horseraddish peroxidase was added to all wells at a concentration of 0.25 μg/ml. The plate was covered with foil and incubated at 37° C. for 1 hour. The plate was washed 5 times with washing buffer using automatic washer. TMB substrate (Seramun, Germany) was added to all wells and the plate was incubated at room temperature for 15 min. Reaction was stopped by 4% H₂SO₄ and absorbance was read at 450/620 nm in an ELISA reader (Tecan). A calibration curve with aMMP-8 antigen (Invent, Germany) was always included in each plate.

The results (aMMP-8 value of each sample) were calculated as follows:
1. The average absorbance of each Standard controls and tested clinical sample was calculated.
2. A regression curve was platted with the average absorbance of Standards (Y-axis) against its corresponding concentration in log (X-axis).
3. The average absorbance of each clinical sample was used to determine the corresponding aMMP-8 concentration by simple interpolation from this standard curve and multiplying by dilution factor (1:50).
4. The samples with average absorbance greater than highest standard were tested again with higher dilution factor.

### Example 5

### Stress Incubation Stability of aMMP-8 Antigen in the GCF Strips

Method: Two “spike samples” were prepared by diluting aMMP-8 antigen in negative clinical sample. The spiked samples were classified as High (40 μg/ml) and Medium (10 μg/ml). 1 μl of each spiked samples was pipetted on strips (n=36), and the strips were placed in 1.5 ml eppendorf tubes. The eppendorf tubes were incubated at 37° C. (n=18) and RT (n=18). The strips after absorbing the aMMP-8 antigen are known as absorbed strips. In addition 1 μl of each spiked samples was directly pipetted in the eppendorf tubes containing elution puffer, and incubated them at 37° C. (n=18) and RT (n=18) as positive control. 1 μl of negative clinical sample (without spiking) was directly pipetted in the eppendorf tubes containing elution puffer, and incubated them at 37° C. (n=18) and RT (n=18) as negative control.

Elution time:

The elution was performed with 2 strips each on Day 0, 1, 4, 7, 15, 21 and 31. The control tubes were also separated in similar manner. The eluted samples and control tubes were stored at −20° C. and tested together at later time period. The eluted samples were tested in ELISA as described above.

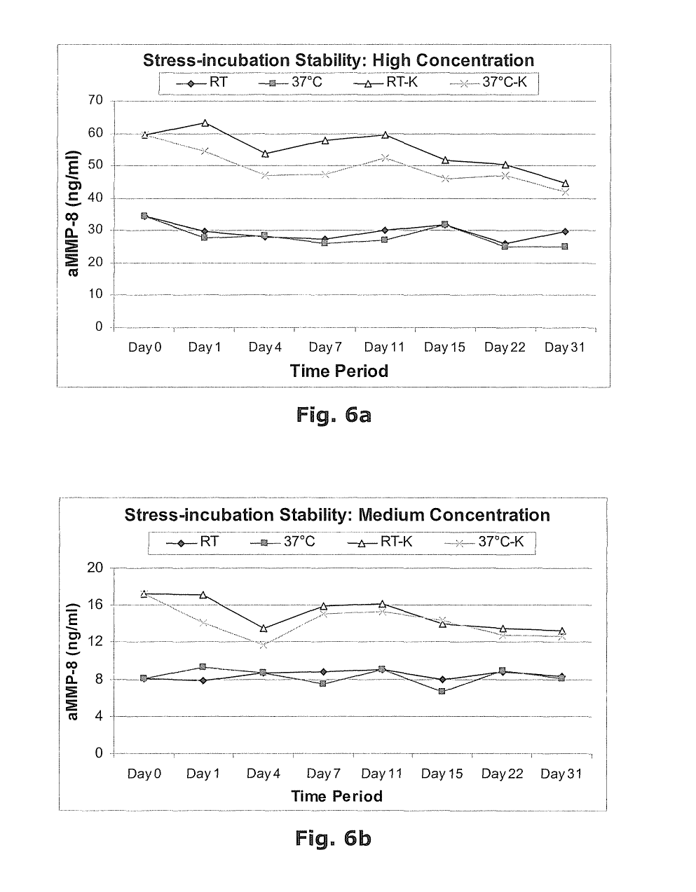

Results: The aMMP-8 concentration (High or medium) in the absorbed strips was stable even after incubation at 37° C. for a period of 31 days (FIGS. 6a and 6b). The FIGS. 6a and 6b describes the concentration of aMMP-8 in the absorbed strips incubated at room temperature (RT) and 37° C., and also the positive controls incubated at room temperature (RT-K) and 37° C. (37° C.-K). There was slight decrease in the concentration in the positive control samples; however the concentration in absorbed strips remained stable. No aMMP-8 was detected in the negative control samples. This indicates that aMMP-8 is highly stable in the absorbed strips and could be used as a medium for preservation or storage or transportation.

### Example 6

### Transport Stability of aMMP-8 Antigen in the GCF Strips

Method: One “spike sample” was prepared by diluting aMMP-8 antigen in negative clinical sample. The spiked samples were classified as Medium (10 μg/ml). Two methods for absorption of strips with aMMP-8 antigen were used.

Absorption method I: 1 μl of spiked samples was pipetted on strips (n=18), and then placed in 1.5 ml eppendorf tubes.

Absorption method II: An artificial model was fabricated in similar fashion as teeth pocket which was named as “Teeth-pocket model”. 1 μl of spiked samples was pipetted in the pockets of the “Teeth-pocket model” and the individual strip was placed in the pocket of the “Teeth-pocket model” in similar manner as used in real conditions. The strips (n=18) were allowed to absorb the spiked sample for 30 s, and then placed in 1.5 ml eppendorf tubes.

In addition, 1 μl of spiked samples was directly pipetted in the eppendorf tubes (n=18) containing elution puffer as positive control. 1 μl of negative clinical sample (without spiking) was directly pipetted in the eppendorf tubes (n=18) containing elution puffer as negative control.

All the absorbed strips and controls were subjected to following conditions pertaining to temperature fluctuation anticipated to occur during transport:
Two strips of both methods were eluted immediately as reference (Day 0). Two controls were also separated as Day 0 reference.
All rest tubes and controls were incubated at room temperature (RT) for 1 hr, 4° C. for 4 hours, 42° C. for 16 hours
Elution from 8 strips was performed as intermediate reference (Day 1). 8 controls tubes were also separated as Day 1 reference.
All rest tubes and controls were incubated at RT for 2 days, 4° C. for 2 days and RT for 2 hr
Elution was performed with all the rest strips. This was considered as final reference (Day 5), end of transportation. Rest of all control tubes were also separated.

The eluted samples and control tubes were stored at −20° C. and tested together. The eluted samples were tested in ELISA as described above.

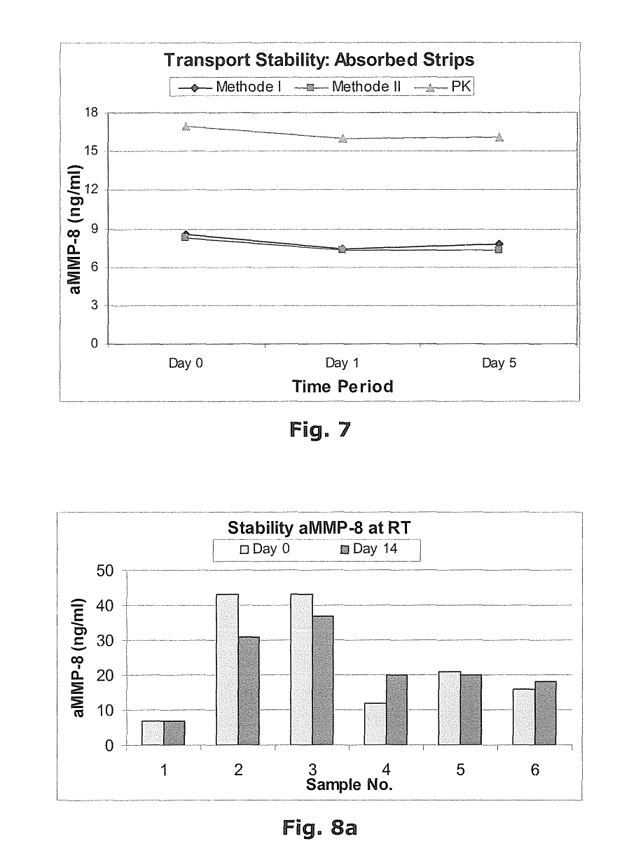

Results: The concentration of aMMP-8 remained stable in the absorbed strips during the temperature fluctuation that might occur during transport of the samples (FIG. 7). FIG. 7 depicts a constant amount of aMMP-8 in the absorbed strips over a period of 5 days with fluctuation in temperature from 4° C. to 42° C. No difference was observed between the two absorption methods. Similar results were found in the positive controls (PK) during the same period; however we have observed from previous experiment that there was slight decrease in the concentration of aMMP-8 in positive controls when kept at room temperature and 37° C. over a period of 31 days. No aMMP-8 was detected in the negative control samples.

### Example 7

### Stability of aMMP-8 in the Absorbed GCF Strips Collected from Patients (Clinical Samples)

### Methods:

1. GCF Collection: The GCF was collected with standard procedures (collection strips) from healthy, gingivitis and periodontitis affected teeth (Munjal et al., 2007; Mäntylä et al., 2003). Four samples were collected from each tooth (same site) for 4 times after a gap of every 30 min. The subjects were advised not to eat or drink 1 hr before and during the period of sample taking.
2. Elution: The strips were either eluted immediately or kept in Eppendorf tube at 4° C. or room temperature (RT) and eluted on days 0, 4, 7 and 14. Each strip eluted at a later time period had a reference at day 0 (immediate elution). The samples (after elution) were divided into different aliquots, and stored at −20° C.
3. The aMMP-8 Recovery assay: The concentration of aMMP-8 in the GCF samples was determined with ELISA as described above.
4. Isoform profiles and collagenase activity: The molecular weight forms of MMP-8 and -13 were detected by a modified ECL Western blotting kit according to protocol recommended by the manufacturer (GE Healthcare, Amersham, UK). Briefly, 14 μl GCF samples were mixed with 5 μl a modified Laemmli's sample buffer without any reducing reagents and heated for 5 min, followed by protein separation with 11% sodium dodecyl sulphate (SDS)-polyacrylamide gels. After electrophoresis the proteins were electrotransferred onto nitrocellulose membranes (Protran, Whatman GmbH, Dassel, Germany). Non-specific binding was blocked with 5% milk powder (Valio Ltd., Helsinki, Finland) in TBST buffer (10 mM Tris-HCl, pH 7.5, containing 22 mM NaCl and 0.05% Triton-X) for 1 h. Then the membranes were washed 3 times for 15 min in TBST and let them be in TBST overnight and then, in the next morning, membranes were incubated with primary antibody polyclonal anti-MMP-8 (1:500) (Hanemaaijer et al. 1997, J Biol Chem 272: 31504-9; Sorsa et al. 1994, Ann NY Acad Sci 732: 112-31) and monoclonal anti-MMP-13 ([1 μl/ml], Calbiochem, A Brand of EMD Biosciences, Inc. La Jolla, Calif., An Affiliate of Merck KGaA, Darmstadt, Germany, Cat #IM44L) for 5 hr. As a secondary antibody was used horseradish peroxidase-linked anti-rabbit IgG for MMP-8 and anti-mouse IgG for MMP-13 (GE Healthcare), for 1 h. Before and after the secondary antibodies, membranes were washed 4 times for 15 min in TBST. The proteins were visualized using the enhanced chemiluminescence (ECL) system (GE Healthcare). The intensity of different molecular weight forms of MMP-8 and -13 were scanned and analyzed using a Bio-Rad Model GS-700 Imaging Densitometer using Quantity One, Basic-program (Bio-Rad Laboratories, Hercules, Calif., USA). Results were expressed as Optical Density/mm² (ODu).
5. Isoform profiles and gelatinase activity: The gelatinolytic activity was assayed with the use of an enzymography 11% sodium dodecyl sulphate (SDS)-polyacrylamide gels impregnated with 1 mg/ml fluorescent dye using 2-methoxy-2,4-diphenyl-3-(2H) furanone (MDPF, Fluka, Buchs SG, Switzerland) labeled gelatin as substrate. Before the electrophoresis the GCF samples (14 μl) were incubated with 5 μl a modified Laemmli's sample buffer without any reducing reagents for 2 hr. Prestained low range molecular weight SDS-PAGE standards (BioRad, Hercules, Calif., USA) were used as molecular weight markers. After electrophoresis, the gels were washed for 30 min with 50 mM Tris-HCl buffer, pH 7.5, containing 2.5% Tween 80, 0.02% NaN₃ and then for 30 min with the same buffer supplemented with 0.5 mM CaCl₂ and 1 μM ZnCl₂. Finally, the gels were incubated in 50 mM Tris-HCl buffer, pH 7.5, containing 0.02% NaN₃, 0.5 mM CaCl₂ and 1 μM ZnCl₂ overnight at 37° C. The degradation of gelatin was visualized under UV light and then stained with 1% Coomassie Brilliant Blue R 250. The gelatinolytic activity was visualized as clear bands against the blue background on stained gels. The intensities of gelatinolytic activity were evaluated with a Bio-Rad Model GS-700 Imaging Densitometer using Quantity One, Basic-program (Bio-Rad Laboratories, Hercules, Calif., USA). Results were expressed as Optical Density/mm² (ODu).
6. Recovery of other enzymes: The PMN elastase and myeloperoxidase (MPO) concentrations were determined using commercially available and enzyme-linked immunosorbent assay (ELISA) kits according to the manufacturer's protocol (PMN elastase; Bender MedSystems GmbH, Campus Vienna Biocenter 2, Vienna, Austria and MPO; Immundiagnostik AG, Stubenwald-Allee 8a, Bensheim, Germany). The so called secondary antibody was conjugated with horseradish peroxidase for PMN elastase and rabbit anti-MPO peroxidase labelled antibody for MPO. The tetra methyl benzidine (TMB) was used as a substrate in all kits. The absorbance was measured at 450 nm using Labsystems Multiskan RC (Thermo Bioanalysis Corporation, Santa FE, USA). PMN elastase and MPO levels were expressed as ng/ml.

### Results:

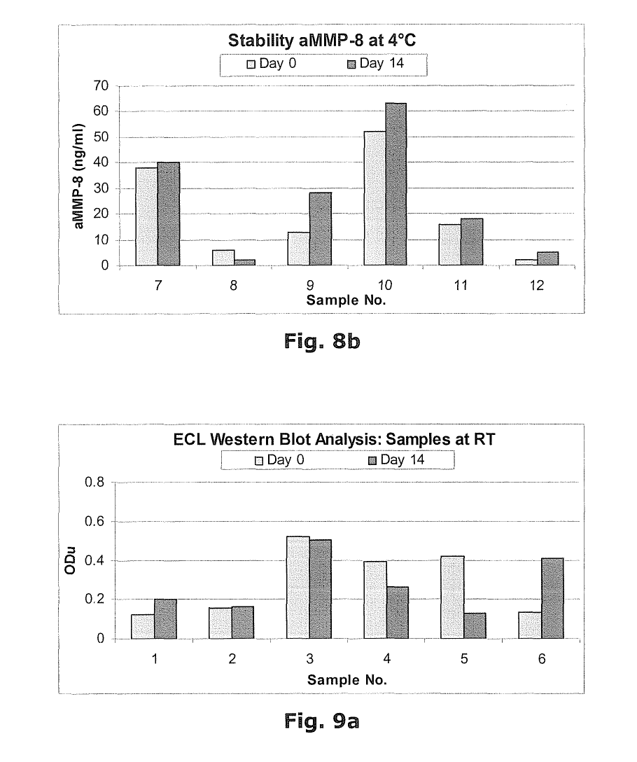

The aMMP-8 Recovery assay: The concentrations of aMMP-8 recovered from the absorbed strips at day 0 and 14 incubated by RT and 4° C. are shown in FIGS. 8a and 8b, respectively. The concentration of aMMP-8 recovered from absorbed strips incubated at 4° C. and RT and eluted on day 0, 4, 7 and 14 were in same range indicating stability of the enzyme in the device over at least a period of 14 days in real conditions.

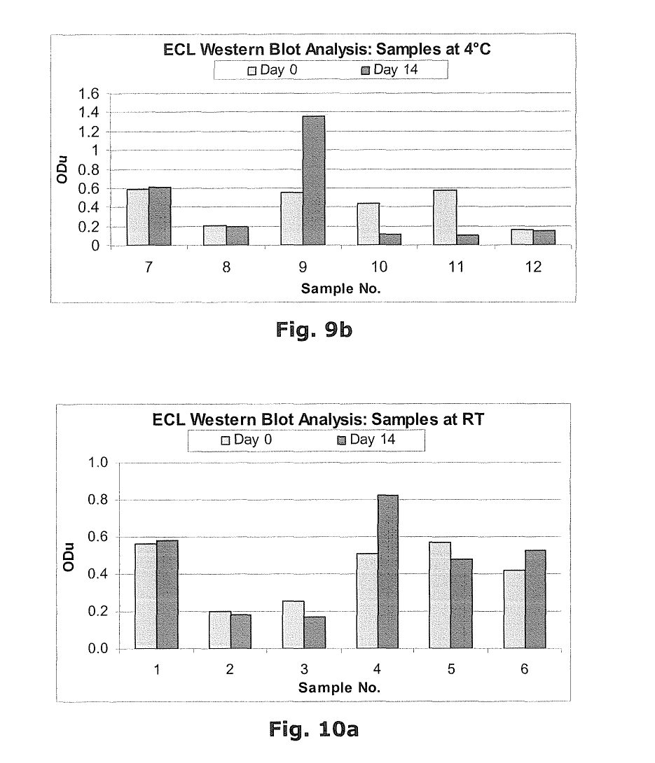

Isoform profile of collagenases and their densitometry quantification: Different molecular weight forms of MMP-8 like PMN type pro-form, fibroblast type pro-form, complex form and PMN type activated form without fragmentation were expressed in western blot analysis. The concentrations of aMMP-8 quantified using densitometry expressed as Optical Density/mm² in the absorbed strips at day 0 and day 14 incubated by RT and 4° C. are shown in FIGS. 9a and 9b, respectively. Similarly, different weight forms of MMP-13 like complex and pro-MMP-form were expressed without fragmentation. The pro-active form was not activated and none of the isoforms were further fragmented during the period of incubation in the absorbed strips. This indicated that the MMP-8 and MMP-13 were stable and intact in the absorbed strips at RT and 4° C. for over a period of 14 days.

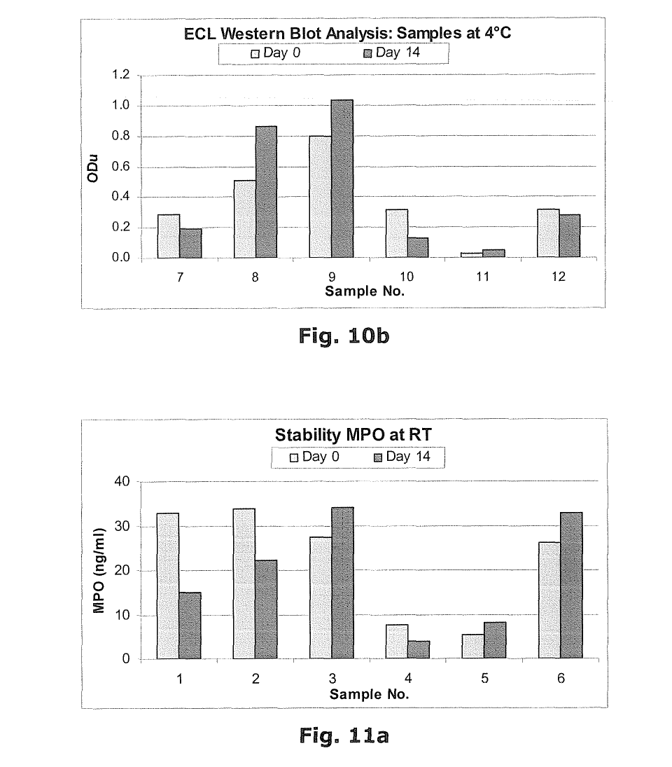

Gelatinase activity and their densitometry quantification: Different molecular weight forms of MMP-9 and 2 like pro-form, complex form and activated form without fragmentation were expressed in western blot analysis. The concentrations of the activated form of MMP-9 quantified using densitometry expressed as Optical Density/mm² in the absorbed strips at day 0 and day 14 incubated by RT and 4° C. are shown in FIGS. 10a and 10b, respectively. This indicated that not only collagenases but also gelatinases activity and their concentration remained stable in the absorbed strips incubated at RT and 4° C. over a period of 14 days.

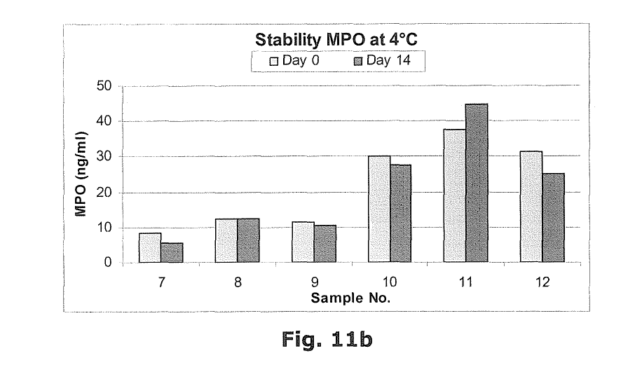

Recovery of other enzymes: The concentrations of myeloperoxidase (MPO) quantified using ELISA in the absorbed strips at day 0 and day 14 incubated by RT and 4° C. are shown in FIGS. 11a and 11b, respectively. The concentration of MPO recovered from absorbed strips incubated at 4° C. and RT and eluted on day 0, 4, 7 and 14 were in same range indicating stability of the enzyme in the strips over at least a period of 14 days in real conditions. Similar results were observed with concentration of Elastase. This clearly indicated that several other enzymes or proteins also remained stable in the absorbed strips.

Next to aMMP-8, Elastase and MPO some other relevant factors for dental diseases are known to be present in GCF. Some of these were qualified with the sample taking system during an Experimental Gingivitis (EG) study design with n=12 subjects: tooth cleaning at day 0 and subsequent no mechanical tooth cleaning (i.e. tooth brushing) during 14 days. The assessment of the biomarkers was performed by collecting GCF with the strips. Samples were eluted immediately and after 48 hours, elution's frozen at −20C and later qualified with state of the art laboratory methods. The proteins assessed were MMP-13, TNFa, Interleukin 1β, Osteoprotegerin (OPG). As to be expected theses parameters were not present in EG conditions in relevant amounts, sometimes they were even below the detection limit of the corresponding ELISA detection test. Therefore a re-assessment was performed with calibrators taken from the individual test kits utilized. As such calibrators (1 μl) were absorbed with the strips n=4 per protein. Two of the strips were eluted immediately after the absorption, two were stored for 48 hours at 4° C. and eluted thereafter. All elutions were performed in the same way: strips were introduced into 300-600 μl of elution buffer for 5 minutes with 5 times 5 shaking cycles within this period of time. After 5 minutes the strips were removed from the buffer and elutions were frozen at −20° C. immediately thereafter. Such a comparison of the strips eluted immediately and of the strips stored for 48 hours at 4° C. was assessable. The results were consistently satisfying for all proteins under observation. The rate of loss of concentration was in the range of 0.3 to 10.9 percent and within the correlation coefficient of the analytical methods utilized. Due to the small number of experiments a trend prognosis can be given that the claim of a conservation capability of the strip with all the proteins assessed is similar to the data found with aMMP-8.

1.-13. (canceled)

14. A planar device for absorbing substances from body fluids, comprising:
a receptor element and a support element wherein
said receptor element is hydrophilic and has a pore size of from 0.22 μm to 5 μm and comprises a plastic material;
said support element is hydrophobic and at least partially covers a first surface of the receptor element; and
the device further comprises a discriminator element situated at a second surface that opposes the first surface of the receptor element.

15. The device of claim 14, wherein said receptor element has a pore size of from 0.6 μm to 2.5 μm.

16. The device of claim 14 wherein said receptor element is a membrane.

17. The device of claim 14 wherein said plastic material is a fluorohydrocarbon polymer.

18. The device of claim 14 comprising a first end and a second end, with the first end being rounded and with the discriminator element being located at the second end.

19. The device of claim 14, wherein the plastic material or mixture of plastic materials has a modulus of elasticity of from 1 to 6 GPa.

20. The device of claim 14, wherein the support element comprises a plastic material with a modulus of elasticity from 1 to 4 GPa.

21. The device of claim 14, where the device has a rounded end at the receptor element and also at the support element.

22. The device according to claim 14 further comprising a marking element which indicates a desired immersion depth of the device in the body fluid.

23. A method of storing a substance comprising:
absorbing the substance with a planar device comprising a receptor element and a support element wherein said receptor element is hydrophilic and has a pore size of from 0.22 μm to 5 μm and comprises a plastic material; said support element is hydrophobic and at least partially covers a first surface of the receptor element; and the device further comprises a discriminator element situated at a second surface that opposes the first surface of the receptor element,
and storing the absorbed substance in the device for a period from 1 to 31 days.

24. The method of claim 23 wherein the substance comprises a protein and/or an enzyme.

25. The method of claim 24 wherein an enzyme is eluted from the absorbed substance and the enzyme is chosen from the group consisting of matrix metalloproteinase-8 (MMP-8), MMP-13, TNFα, Interleukin 1β (IL-1β), and osteoprotegerin.

26. The method of claim 23 wherein enzymes are eluted from the absorbed substance and the enzymes are stable over a storage time of from 1 to 31 days at a temperature of from 4° C. to 42° C.

27. The method of claim 26 wherein activity of the enzymes is approximately maintained after the storage.

28. The method of claim 24 wherein an protein is eluted from the absorbed substance and the epitopes of the proteins are substantially maintained after the storage.

29. The method of claim 23 wherein the substance is a bodily fluid chosen from the group consisting of gingival crevicular fluid or lacrimal fluid.
